(19) United States
(12) Patent Application Publication
(10) Pub. No.: US 2025/0040555 A1
(43) Pub. Date: Feb. 6, 2025

(54) An Antimicrobial Disinfectant Composition for Air Diffusion for Use in an Air Diffuser

(71) Applicant: Ah Eng SIAW, Johor Bahru (MY)

(72) Inventor: AH ENG SIAW, SINGAPORE (SG)

(21) Appl. No.: 18/715,459

(22) PCT Filed: Nov. 30, 2022
(86) PCT No.: PCT/MY2022/050124

§ 371 Date: May 31, 2024

(30) Foreign Application Priority Data
Dec. 2, 2021 (MY) PI2021007209

(51) Int. Cl. A01N 59/20; A01N 25/06; A01P 1/00; A61L 9/01; A61L 9/14

(52) U.S. Cl. CPC A01N 59/20; A01N 25/06; A01P 1/00; A61L 9/01; A61L 9/14

(57) ABSTRACT

The invention relates to an antimicrobial disinfectant composition for use in an air diffuser and a method for preparing thereof. Particularly, the antimicrobial disinfectant composition comprises a nano-sized particle selected from the group consisting of elemental metal, metal oxide, metal salt, colloidal metal, metal ion, or a combination thereof and a liquid carrier. Preferably, the atoms of the nano-sized particle are in a state of energy excitation that vibrate at a frequency of 1 kHz to 1 MHz for a period of time upon being subjected to bombardment with a vibration force at the frequency for at least 6 hours. Advantageously, the antimicrobial disinfectant composition can eliminate a variety of microbes. In addition, the atoms hold sufficient vibration energy for a period of time and are able to transfer the vibration energy to surrounding microbes to facilitate the disinfection and improve the disinfection efficacy.

### FIELD OF INVENTION

The present invention relates to an antimicrobial disinfectant composition for use in an air diffuser. Particularly, the antimicrobial disinfectant composition comprises atoms that are in a state of energy excitation for a period of time.

### BACKGROUND OF THE INVENTION

Conventional antimicrobial air diffusion treatment technology is based on dispersing antimicrobial particles into air to eliminate airborne microbes. Some examples are vanadium, copper, zinc, rhodium, silver, and nickel. Great attention has been focused on using nano-sized silver and nano-sized copper particles in a disinfectant for disinfecting the surface.

Silver and copper can provide natural anti-bacterial, anti-viral, and anti-fungal benefits. For example, Mathur et al. (doi: 10.1080/21691401.2017.1414825) discussed on the antimicrobial mechanism of silver. When the nano-sized silver particles come into contact with bacteria or viruses, they suppress the cells' nutrient transport, attack the cell membrane and interfere with cell division to hinder the reproduction of the germs. Nano-sized copper particles, on the other hand, damage bacterial cell membranes or “envelopes” and can destroy the DNA or RNA of the microbe. Nano-sized copper particles generate oxidative stress on bacterial cells and create hydrogen peroxide that can kill the cells. They also interfere with proteins that play vital functions. However, the conventional technology requires a direct contact of the antimicrobial particles with the airborne microbes thereby limiting the efficacy of the technology.

Therefore, it is desirable to provide an antimicrobial disinfectant composition that is capable of providing enhanced antimicrobial functionality. Desirably, the improved antimicrobial disinfectant composition is capable of eliminating or reducing microbes without directly contacting thereof. The present invention provides a solution to the aforementioned problems.

### SUMMARY OF THE INVENTION

One object of the present invention is to provide an antimicrobial disinfectant composition for use in an air diffuser comprising a nano-sized particle selected from the group consisting of elemental metal, metal oxide, metal salt, colloidal metal, metal ion, or a combination thereof to provide antimicrobial functionality. Advantageously, the antimicrobial disinfectant composition can eliminate a variety of microbes.

Another object of the present invention is to provide an antimicrobial disinfectant composition for use in an air diffuser which atoms of the nano-sized particle are in a state of energy excitation that vibrate for a period of time upon being subjected to bombardment with a vibration force. Advantageously, the atoms hold sufficient vibration energy for a period of time and are able to transfer the vibration energy to surrounding microbes to facilitate the disinfection and improve the disinfection efficacy.

At least one of the preceding objects is met, in whole or in part, by the present invention, in which the embodiment of the present invention describes an antimicrobial disinfectant composition for use in an air diffuser comprising a nano-sized particle; and a liquid carrier.

According to a preferred embodiment of the present invention, atoms of the nano-sized particle are in a state of energy excitation that vibrate at a frequency of 1 kHz to 1 MHz for a period of time upon being subjected to bombardment with a vibration force at the frequency for at least 6 hours.

Preferably, the metal oxide is silver oxide, copper oxide, titanium oxide, or a combination thereof.

Preferably, the nano-sized particle is present at about 0.001% to about 10.0% by weight of the antimicrobial disinfectant composition.

Preferably, the nano-sized particle has a particle size ranging from about 2 nm to about 100 nm.

Another embodiment of the present invention describes a method for preparing an antimicrobial disinfectant composition for use in an air diffuser comprising the steps of providing a mixture comprising a nano-sized particle selected from the group consisting of elemental metal, metal oxide, metal salt, colloidal metal, metal ion, or a combination thereof and a liquid carrier; and subjecting the mixture to a bombardment with a vibration force such that atoms of the nano-sized particle are in a state of energy excitation that vibrate at a frequency of 1 kHz to 1 MHz for a period of time.

Yet, another embodiment of the present invention describes a use of the antimicrobial disinfectant composition in an air diffuser.

### BRIEF DESCRIPTION OF THE DRAWINGS

For the purpose of facilitating an understanding of the invention, there is illustrated in the accompanying drawings the preferred embodiments from an inspection of which when considered in connection with the following description, the invention, its construction and operation and many of its advantages will be readily understood and appreciated.

FIG. 1 shows an experimental setup to evaluate antimicrobial functionality of the antimicrobial disinfectant composition

### DETAILED DESCRIPTION OF THE INVENTION

One skilled in the art will readily appreciate that the invention is well adapted to carry out the objects and obtain the ends and advantages mentioned, as well as those inherent therein. The embodiment described herein is not intended as limitations on the scope of the invention.

The invention discloses an antimicrobial disinfectant composition for use in an air diffuser. By way of examples, the air diffuser is a humidifier or a mist dispenser.

In one embodiment of the present invention, the antimicrobial disinfectant composition comprises a nano-sized particle selected from the group consisting of elemental metal, metal oxide, metal salt, colloidal metal, metal ion, or a combination thereof and a liquid carrier. The nano-sized particle is capable of slowing or hindering microbial growth and/or inactivating or killing microbes. Preferably, the elemental metal is silver, copper, or a combination thereof. Preferably, the metal oxide is silver oxide, copper oxide, or a combination thereof. Preferably, the metal salt is silver sulphate, copper sulphate, or a combination thereof. Preferably, the colloidal metal is colloidal silver, colloidal copper, or a combination thereof. Preferably, the metal ion is silver ion, copper ion, or a combination thereof. The liquid carrier may be water.

Preferably, the nano-sized particle is present at about 0.001% to about 10.0% by weight of the antimicrobial disinfectant composition. Although a higher concentration of the nano-sized particle can be used, it does not appear to provide notable improvement to the antimicrobial functionality of the antimicrobial disinfectant composition.

Preferably, the nano-sized particle has a particle size ranging from about 2 nm to about 100 nm. A smaller particle side is more preferably as it provides a larger surface-area-to-volume ratio thereby enhancing the efficacy thereof.

In another embodiment of the present invention, atoms of the nano-sized particle are in a state of energy excitation that vibrate at a frequency of 1 kHz to 1 MHz for a period of time upon being subjected to bombardment with a vibration force at the frequency for at least 6 hours. Preferably, nano-sized particle derived from a high electrical conductivity metal is used because it can hold a higher charge and therefore, a higher capacity to hold vibration energy. The vibration energy does not dissipate after the bombardment with vibration force is halted.

The cell wall or cell membrane of a microbe has a specific natural frequency in which upon vibrated at such frequency will cause it to shatter or break. This phenomenon can be observed when a glass is being subjected to a certain pitch which is equal to its natural frequency. As such, the frequency is selected based on natural frequency of microbes. Advantageously, atoms of the nano-sized particle can be subjected to a broad range of frequency as abovementioned such that it can be used against a wider variety of microbes.

Advantageously, the antimicrobial disinfectant composition does not require to come into direct contact with the microbes to provide antimicrobial functionality. When in use, the antimicrobial disinfectant composition is being diffused into the air through the use of an air diffuser as abovementioned. Then, the atoms of the nano-sized particle transfer the vibration energy in all direction at a distance ranging at about 10 mm to about 20 mm to the microbes from the atoms. In this process, the cell membranes or cell walls of the microorganisms are induced to vibrate at a similar frequency. Particularly, the microbial cell membranes or cell walls are vibrating at their natural frequency where resonance occurs which eventually causing them to shatter and break apart. In some embodiments of the present invention, the antimicrobial disinfectant composition is capable of reducing microbes after 5 minutes of application. It should be emphasized that such application does not involve direct contact between the nano-sized particle and microbes.

Further embodiment of the present invention describes a method for preparing the antimicrobial disinfectant composition for use in an air diffuser. The method provides a step of providing a mixture comprising a nano-sized particle selected from the group consisting of elemental metal, metal oxide, metal salt, colloidal metal, metal ion, or a combination thereof and a liquid carrier. Preferably, the nano-sized particle and the liquid carrier are homogeneously mixed together. More preferably, a homogenizer is used at a speed of 1,000 rotation per minute (rpm) to 12,000 rpm for at least 30 minutes.

Subsequently, the method provides a step of subjecting the mixture to a bombardment with a vibration force such that atoms of the nano-sized particle are in a state of energy excitation that vibrate at a frequency of 1 kHz to 1 MHz for a period of time. Preferably, the bombardment with a vibration force is performed by means of ultrasonication. A sufficiently long period of bombardment time is required in order to allow atoms of the nano-sized particle to capture and hold the vibration energy from the vibration force for a period of time. In this process, the atoms are excited to vibrate vigorously for a period of time at a frequency similar to the frequency of the vibration force.

The present invention further discloses a use of the antimicrobial disinfectant composition in an air diffuser. When in use, the antimicrobial disinfectant composition is placed in the air diffuser such that the antimicrobial disinfectant composition is dispersed therefrom to the air. Then, the atoms of the nano-sized particle vibrate at a frequency that destroys microbes in the vicinity having a natural frequency that matches the vibration frequency of the atoms.

Although the present invention has been described and illustrated in detail, it is to be understood that the same is by the way of illustration and example, and is not to be taken by way of limitation. The scope of the present invention is to be limited only by the terms of the appended claims.

### EXAMPLES

An example is provided below to illustrate different aspects and embodiments of the present invention. The example is not intended in any way to limit the disclosed invention, which is limited only by the claims.

### Example 1

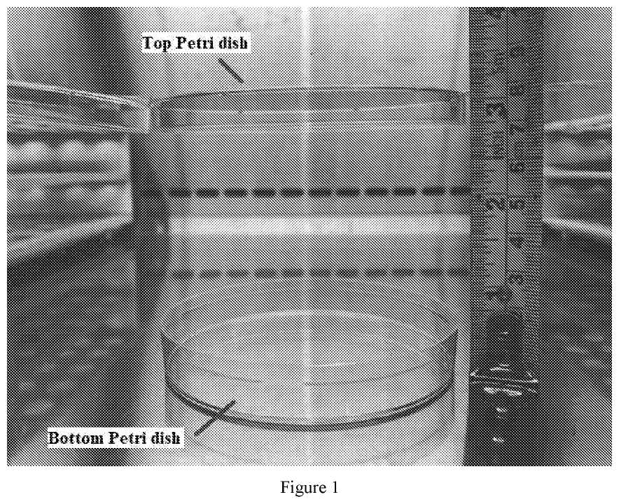

An experiment was designed to evaluate antimicrobial functionality of the antimicrobial disinfectant composition. The experimental setup is shown in FIG. 1. An inoculum comprising a concentration of 10⁷-10⁸ colony forming unit (CFU) of Escherichia coli was inoculated on a sterile Petri dish, referred as “bottom Petri dish” hereinafter. The bottom Petri dish is allowed to dry at 35° C. for 1 hour. The antimicrobial disinfectant composition is applied on another sterile Petri dish, referred as “top Petri dish” hereinafter, placed above the bottom Petri dish at a distance of 3 inches. The bottom Petri dish was exposed to the top Petri dish for 5 minutes, 10 minutes, and 30 minutes. After each exposure, the bottom Petri dish is extracted for microbiological enumeration test to obtain a treated count.

Table 1 shows results of microbial reduction percentage obtained from the experiment. Accordingly, an exposure time of 5 minutes shows microbial reduction of 76%. It should be emphasized that there was no direct contact between the antimicrobial disinfectant composition or the top Petri dish and the microbes or the bottom Petri dish. Therefore, it can be concluded that the vibration energy of the atoms of the nano-sized metal oxide in the antimicrobial disinfectant composition vibrates at a frequency which matches the natural frequency of the microbes thereby destroying the cell walls thereof and resulted in a reduction in the amount of microbes.

TABLE 1
Results of microbial reduction percentage.

| Exposure time | Mean untreated | Mean treated | Microbial |
|---|---|---|---|
| (minutes) | count (CFU) | count (CFU) | reduction (%) |
| 5 | 2,000,000 | 480,000 | 76.0 |
| 10 | 2,000,000 | 510,000 | 74.5 |
| 30 | 2,000,000 | 610,000 | 69.5 |

1.-10. (canceled)

11. An antimicrobial disinfectant composition for use in an air diffuser, comprising:
a nano-sized particle selected from the group consisting of elemental metal, metal oxide, metal salt, colloidal metal, metal ion, and combinations thereof; and
a liquid carrier, wherein atoms of the nano-sized particle are in a state of energy excitation that vibrate at a frequency of 1 kHz to 1 MHz for a period of time upon being subjected to bombardment with a vibration force at the frequency for at least 6 hours.

12. The antimicrobial disinfectant composition according to claim 11, wherein the metal oxide is selected from the group consisting of silver oxide, copper oxide, and combinations thereof.

13. The antimicrobial disinfectant composition according to claim 11, wherein the nano-sized particle is present at about 0.001% to about 10.0% by weight of the antimicrobial disinfectant composition.

14. The antimicrobial disinfectant composition according to claim 11, wherein the nano-sized particle has a particle size ranging from about 2 nm to about 100 nm.

15. A method for preparing an antimicrobial disinfectant composition for use in an air diffuser, comprising the steps of:
providing a mixture comprising a nano-sized particle selected from the group consisting of elemental metal, metal oxide, metal salt, colloidal metal, metal ion, and combinations thereof, and a liquid carrier; and
subjecting the mixture to a bombardment with a vibration force for at least 6 hours such that atoms of the nano-sized particle are in a state of energy excitation that vibrate at a frequency of 1 kHz to 1 MHz for a period of time.

16. The method according to claim 15, wherein the metal oxide is selected from the group consisting of silver oxide, copper oxide, and combinations thereof.

17. The method according to claim 15, wherein the nano-sized particle is present at about 0.001% to about 10.0% by weight of the antimicrobial disinfectant.

18. The method according to claim 15, wherein the nano-sized particle has a particle size ranging from about 2 nm to about 100 nm.

19. Use of the antimicrobial disinfectant composition according to claim 11 in an air diffuser.
